(19) United States
(12) Patent Application Publication
(10) Pub. No.: US 2024/0376065 A1
(43) Pub. Date: Nov. 14, 2024

(54) THIONOLACTONES, PROCESSES OF SYNTHESIS, USES AS COMONOMERS AND FOR POLYMER FUNCTIONALIZATION AND DEGRADATION

(71) Applicants: SPECIALTY OPERATIONS FRANCE, Lyon (FR); Université d'Aix-Marseille, Marseille (FR); Centre National de la Recherche Scientifique, Paris (FR)

(72) Inventors: Pierre-Emmanuel Dufils, Paris (FR); Christopher Mark Plummer, Lodz (PL); David James Wilson, Coye-La-Forêt (FR); Didier Gigmes, Allauch (FR); Yohann Guillaneuf, Marseille (FR)

(73) Assignees: SPECIALTY OPERATIONS FRANCE, Lyon (FR); Université d'Aix-Marseille, Marseille (FR); Centre National de la Recherche Scientifique, Paris (FR)

(21) Appl. No.: 18/293,303

(22) PCT Filed: Jul. 21, 2022
(86) PCT No.: PCT/EP2022/070510

§ 371 Date: Jan. 29, 2024

(30) Foreign Application Priority Data
Jul. 29, 2021 (EP) 21315131.9

(51) Int. Cl. C07D 319/08; C08G 75/26

(52) U.S. Cl. CPC C07D 319/08; C08G 75/26

(57) ABSTRACT

The instant invention relates to thionolactone compounds useful as comonomers to introduce weak bonds for polymer degradation or functionalization. The invention is also directed to processes for the preparation of said compounds, notably by thionation of the corresponding lactones. The invention is furthermore directed to uses of said thionolactones as comonomers, copolymers made from these comonomers, and processes for the preparation of said copolymers.

The instant invention relates to thionolactone compounds useful as comonomers to introduce weak bonds for polymer degradation or functionalization. The invention is also directed to processes for the preparation of said compounds, notably by thionation of the corresponding lactones. The invention is furthermore directed to uses of said thionolactones as comonomers, copolymers made from these comonomers, and processes for the preparation of said copolymers.

Synthetic polymers are usually too big to be degradable as such and could be more easily degraded by reducing their molecular weight. The reduction of the molecular weight can be done by introducing weak linkage in the polymer backbone. These weak linkage can be introduced by copolymerizing usual monomers with compounds like cyclic ketene acetal, or thionolactone. The introduction of weak bonds in synthetic polymers can be achieved by doing radical Ring Opening Polymerization of standards monomers with cyclic monomers. The publication in the Chemical Reviews Chem. Rev. 2017, 117, 3, 1319-1406 is listing many types of monomers that could be used in rROP.

Some thionolactones are known to be used for radical Ring Opening Polymerization (rROP). The most investigated one is dibenzo[c,e]oxepane-5-thione (DOT), which is studied in Bingham, N. M.; Roth, P. J., Degradable vinyl copolymers through thiocarbonyl addition—ring-opening (TARO) polymerization. Chemical Communications 2019, 55 (1), 55-58, in Smith, R. A.; Fu, G.; McAteer, O.; Xu, M.; Gutekunst, W. R., Radical Approach to Thioester-Containing Polymers. Journal of the American Chemical Society 2019, 141 (4), 1446-1451, and in Spick, M. P.; Bingham, N. M.; Li, Y.; de Jesus, J.; Costa, C.; Bailey, M. J.; Roth, P. J., Fully Degradable Thioester-Functional Homo- and Alternating Copolymers Prepared through Addition-Ring-Opening Radical Polymerization. Thiocarbonyl RAFT Macromolecules 2020, 53 (2), 539-547; and in Bingham N. M.; un Nisa, Qamar; Chua, S. H. L., Fontugne, L., Spick, M. P., Roth, P. J., Thioester-Functional Polyacrylamides: Rapid Selective Backbine Degradation Triggers Solubility Switch Based on Aqueous Lower Critical Solution Temperature/Upper Critical Solution Temperature. ACS Applied Polymer Materials 2020, 2, 3400-3449.

Existing thionolactones, and in particular DOT, are not easily copolymerized with usual comonomers and are not hydrosoluble.

The present invention attempts to overcome these drawbacks.

### BRIEF DESCRIPTION

In the framework of an intensive research, the inventors have found new compounds, notably thionolactones, which structure makes them easily copolymerized and more hydrosoluble.

The invention thus relates to those new compounds, notably thionolactones, their processes of preparation, their copolymerization via radical process with usual comonomers and the degradation under specific conditions of the obtained copolymers. The new compounds, notably thionolactones, are also valuable as comonomers for introducing functionality and imparting degradation properties to copolymers obtained therefrom, which is useful for various applications.

In particular, the present invention relates to the following items:

A thionolactone of formula I

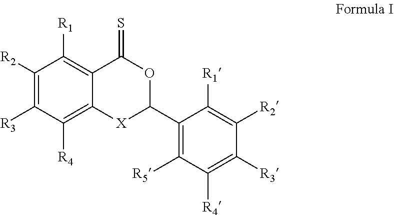

wherein
X is either a heteroatom selected from the group consisting of O and S, or NAlk, with Alk being a linear or branched alkyl group comprising 1 to 6 carbons, preferably comprising 1 to 4 carbons,
R₁, R₂, R₃ and R₄, as well as R₁′, R₂′, R₃′, R₄′ and R₅′ are independently selected from the group consisting of H, halogen, hydroxyl (—OH), thio (—SH), nitro group (—NO₂), amines (—NH₂), ammonium (—NH₄⁺), sulfate (—SO₄⁻), sulfonate (—SO₃⁻), phosphate (—PO₄²⁻), phosphonate (—PO₃²⁻), and hydrocarbyl comprising from 1 to 50 carbon atoms which can be optionally substituted by one or more heteroatom containing groups and/or interrupted by one or more heteroatoms or heteroatom containing groups and/or which can optionally form a cycle, aromatic or not, wherein heteroatom means O, N or S.

A process for the preparation of a thionolactone of formula I as defined herein, by reacting a compound of formula II

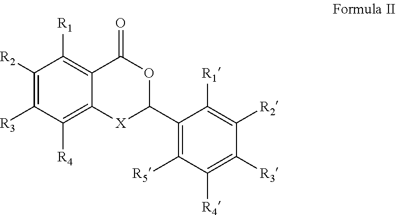

wherein X, R₁, R₂, R₃ and R₄, as well as R₁′, R₂′, R₃′, R₄′ and R₅′ are as defined for formula I
with a thionation agent.

Use of a thionolactone of formula I as defined herein, as comonomer for the preparation of copolymers.

Copolymer comprising the repeating unit of formula III

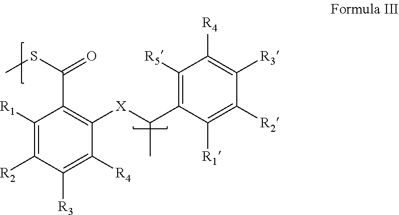

wherein X, R₁, R₂, R₃ and R₄, as well as R₁′, R₂′, R₃′, R₄′ and R₅′ are as defined for formula I,
and at least another repeating unit.

Process for the preparation of a copolymer as defined herein, wherein a thionolactone of formula I is copolymerized with at least one other comonomer selected from the group consisting of alkyl acrylates, N-alkylacrylamides, N,N-dialkylacrylamides, styrene and styrene derivatives, preferably 4-substituted styrene derivatives, acrylonitrile, and mixtures thereof, wherein “alkyl” means a linear or branched alkyl group comprising 1 to 12, preferably 1 to 6 carbons, and wherein the at least one other comonomer is preferably selected from N,N-dimethylacrylamide, styrene and methyl acrylate.

Use of a thionolactone of formula I as defined herein, as comonomer to impart degradability properties to a copolymer at least partially made therefrom.

Process for degradation of a copolymer as defined herein, wherein said copolymer is reacted with a degradation reagent selected from the group consisting of a base, an amine and an oxidant, preferably a base, in an organic or aqueous medium.

Use of a thionolactone of formula I as defined herein, as comonomer to introduce functionality into a copolymer partially made therefrom.

Process for functionalization of a copolymer as defined herein, wherein said copolymer is reacted with a degradation reagent selected from the group consisting of a base, an amine and an oxidant, preferably a base, in an organic or aqueous medium.

Oligomers obtainable by the process for degradation of a copolymer as defined herein or by the process for functionalization of a copolymer as defined herein.

### DETAILED DESCRIPTION

### The Compounds of the Present Invention

The present invention relates to thionolactone compounds of the following formula I

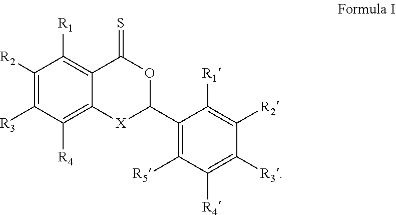

In the compound of Formula I, the residues X, R₁-R₄ and R′₁-R′₅ have the following meaning:
X is either a heteroatom selected from the group consisting of O and S, or NAlk.

The abbreviation “Alk” in the group “NAlk” means a linear or branched alkyl group comprising 1 to 6 carbons, preferably 1 to 4 carbons. Exemplary alkyl groups for use as “Alk” in the compounds of the present invention include, but are not limited to: methyl, ethyl, n-propyl, iso-propyl, cyclo-propyl, n-butyl, iso-butyl, tert-butyl, n-pentyl, iso-pentyl, cyclo-pentyl, n-hexyl, with methyl, ethyl, n-propyl, iso-propyl, n-butyl, iso-butyl, tert-butyl being preferred.

In a preferred embodiment of the compounds of formula I, X is O.

R₁, R₂, R₃ and R₄, as well as R₁′, R₂′, R₃, R₄′ and R₅′ are independently selected from the group consisting of H, halogen, hydroxyl (—OH), thio (—SH), nitro group (—NO₂), amines (—NH₂), ammonium (—NH₄⁺), sulfate (—SO₄⁻), sulfonate (—SO₃⁻), phosphate (—PO₄²⁻), phosphonate (—PO₃²⁻), and hydrocarbyl comprising from 1 to 50 carbon atoms.

The term “hydrocarbyl” as used herein means aryl groups and linear and branched aliphatic groups with 1 to 50 carbon atoms, preferably comprising 1 to 36 carbon atoms, more preferably comprising 1 to 24 carbon atoms, even more preferably comprising 1 to 12 carbons atoms, particularly preferably comprising 1 to 6 carbon atoms, which can be optionally substituted by one or more heteroatom containing groups and/or interrupted by one or more heteroatoms or heteroatom containing groups and/or can optionally form a cycle, said cycle being aromatic or not.

Exemplary linear and branched aliphatic groups useful as R₁, R₂, R₃ and R₄, as well as R₁′, R₂′, R₃′, R₄′ and R₅′ include alkyl groups, such as e.g. methyl, ethyl, n-propyl, iso-propyl, n-butyl, iso-butyl, tert-butyl, n-pentyl, n-hexyl.

“Heteroatom” in the context of the hydrocarbyl group means O, N or S. “Heteroatom containing groups” in the context of the hydrocarbyl group comprise nitrogen containing groups, such as primary, secondary, and tertiary amines, as well as oxygen containing groups, such as carboxy groups, hydroxy groups and ether groups, as well as sulfur containing groups, such as thio groups and thioether groups.

The hydrocarbyl group can optionally form a cycle, said cycle being aromatic or not (i.e. non-aromatic). The cycles can be interrupted by interrupted by one or more heteroatoms or heteroatom containing groups as described above. In that case, the cycle is a heterocycle that can be aromatic or not.

Examples for non-aromatic cycles include cyclo-propyl, cyclo-pentyl, cyclo-hexyl, cyclo-heptyl, cyclo-octyl.

An example for an aromatic cycle is phenyl, which can optionally be further substituted by one or more functional groups, such as
hydrogen or a C₁-C₂₄ linear or branched hydrocarbon group having 1 to 24 carbon atoms which can be optionally substituted and/or interrupted by one or more heteroatoms or heteroatom containing groups,
halogen,
hydroxy (—OH) or alkoxy group (—OR) wherein R is a linear or branched hydrocarbon group having 1 to 24 carbon atoms which can be optionally substituted and/or interrupted by one or more heteroatoms or heteroatom containing groups,
amino group (—NRR′) wherein R and R′ independently represent hydrogen or a linear or branched hydrocarbon group having 1 to 24 carbon atoms which can be optionally substituted and/or interrupted by one or more heteroatoms or heteroatom containing groups,
acyl group (—(C=O)—R) wherein R represents hydrogen or a C₁-C₂₄ linear or branched hydrocarbon group having 1 to 24 carbon atoms which can be optionally substituted and/or interrupted by one or more heteroatoms or heteroatom containing groups,
carboxyl (—COOH) or alkoxycarbonyl group (—(C=O)—OR) wherein R represents a C₁-C₂₄ linear or branched hydrocarbon group having 1 to 24 carbon atoms which can be optionally substituted and/or interrupted by one or more heteroatoms or heteroatom containing groups,
carbamoyl group (—(C=O)—NRR′) wherein R and R′ independently represent hydrogen or a linear or branched hydrocarbon group having 1 to 24 carbon atoms which can be optionally substituted and/or interrupted by one or more heteroatoms or heteroatom containing groups,
alkylsulfonyl group (—SO₂—R) or alkylsulfinyl group (—SO—R) or alkylthio group (—S—R) wherein R represents a C₁-C₂₄ linear or branched hydrocarbon group having 1 to 24 carbon atoms which can be optionally substituted and/or interrupted by one or more heteroatoms or heteroatom containing groups.

Examples for heteroaromatic rings include pyridyl, furanyl, pyrrolyl, thiophenyl, pyrazolyl, imidazolyl, benzimidazolyl, indolyl, quinolinyl, isoquinolinyl, purinyl, pyrimidinyl, thiazolyl, pyrazinyl, pyridazinyl, oxazolyl and triazolyl, which can optionally be further substituted by one or more functional groups as described above for phenyl.

Examples for non-aromatic heterocycles include pyrroldinyl, tetrahydrofuranyl, tetrahydrothiophenyl, piperidinyl, tetrahydropyranyl, tetrahydrothiopyranyl, hexamethyleniminyl, hexamethylenoxidyl, hexymethylensulfidyl, which can optionally be further substituted by one or more functional groups as described above for phenyl.

Preferably, R₁, R₂, R₃ and R₄, as well as R₁′, R₂′, R₃′, R₄′ and R₅′ in the compound of Formula I are independently selected in the group consisting of —H, —F, —Cl, —Br, hydroxyl (—OH), thio (—SH), nitro group (—NO₂), amines (—NH₂), ammonium (—NH₄⁺), sulfate (—SO₄⁻), sulfonate (—SO₃⁻), and hydrocarbyl comprising from 1 to 50 carbon atoms, preferably comprising 1 to 36 carbon atoms, more preferably comprising 1 to 24 carbon atoms, even more preferably comprising 1 to 12 carbons atoms, even more preferably comprising 1 to 6 carbon atoms, substituted by one or more heteroatom containing groups selected from —OY, —NHY, —NY₂, —SY and/or interrupted by one or more heteroatoms or heteroatom containing groups selected from —O—, —NY—, —S—, wherein Y means H or branched or linear alkyl comprising 1 to 12 carbons, preferably linear alkyl comprising 1 to 6 carbons.

In a preferred embodiment, R₁, R₂, R₃, R₄, R₁′, R₂′, R₃′, R₄′ or R₅′, preferably R₃ or R₃′, for example represents a polyethylene glycol group of formula —(CH₂—CH₂—O)ₙ—H with n being an integer varying from 1 to 25, preferably from 1 to 18 and more preferably from 1 to 12. This embodiment is preferably combined with the embodiment, wherein X=O.

In a further preferred embodiment, at least R₁, R₁′, and R₅′ are H in the compounds of formula I. This embodiment is preferably combined with the embodiment, wherein X=O.

More preferably, at least R₁, R₄, R₁′, and R₅′ are H in the compounds of formula I. This embodiment is preferably combined with the embodiment, wherein X=O.

Even more preferably, at least R₁, R₃, R₄, R₁′, R₃′ and R₅′ or at least R₁, R₂, R₄, R₁′, R₂′, R₄′ and R₅′ are H in the compounds of formula I. This embodiment is preferably combined with the embodiment, wherein X=O.

Particularly preferably, R₁, R₂, R₃ and R₄, as well as R₁′, R₂′, R₃′, R₄′ and R₅′ are H in the compounds of formula I. This embodiment is preferably combined with the embodiment, wherein X=O.

In a very preferred embodiment, R₁, R₂, R₃ and R₄, as well as R₁′, R₂′, R₃′, R₄′ and R₅′ are thus H and X is O in the compounds of formula I. Said compound is 2-phenyl-4H-benzo[d][1,3]dioxine-4-thione.

The thionolactone compounds of formula I are suitable for use as comonomers for the provision of copolymers. These copolymers comprise weak thioester linkages derived from the thionolactone moiety of the compounds of formula I that make the resulting copolymers useful for degradation and functionalization purposes.

### Process for the Preparation of a Compound of Formula I

The present invention furthermore relates to a process for the preparation of a thionolactone of formula I. Said process is a thionation reaction, wherein a compound of the following formula II

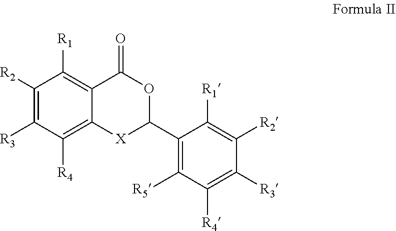

is reacted with a thionation agent.

In the compound of formula II, X, R₁, R₂, R₃ and R₄, as well as R₁′, R₂′, R₃′, R₄′ and R₅′ are defined as described herein above in detail for the compound of formula I.

The thionation agent used in the process of the present invention for the preparation of the compounds of formula I is typically selected from the group consisting of Lawesson's reagent (2,4-bis(4-methoxyphenyl)-1,3,2,4-dithiadiphosphetane 2,4-disulfide), Davy's reagent (2,4-Bis(methylthio)-2,4-dithioxo-1,3,2,4-dithiadiphosphetane, (CH₃S)₂P₂S₄)), Curphey's reagent (hexamethyldisiloxane (HMDO)/phosphorus pentasulfide (P₄S₁₀)), Kaushik's reagent (P₄S₁₀/Al₂O₃), Bernthsen reagent (S₈/I₂), Heimgartner's reagent (2,4-bis(4-methylphenylthio)-1,3,2λ5,4λ5-dithiadiphosphetane-2,4-dithione), Jan Bergman Reagent (for reference, see: http://vironovamedical.com/wp-content/uploads/2019/04/Vironova-Medical-Thionation-181029.pdf), Belleau reagent (2,4-bis(4-phenoxyphenyl)-1,3,2,4-dithiadiphosphetane 2,4-disulfide), Japanese reagent (2,4-bis(phenylthio)-1,3,2,4-dithiadiphosphetane 2,4-disulfide), H₂S, CS₂, R₂PSX, (Et₂Al)₂S, NaSH, TMS₂S, thiourea/Ru (III)/Al₂O₃, benzyltriethylammonium tetrathiomolybdate, thioacyl-N-pthalimides, elemental sulfur, aqueous ammonium sulfide, SiS₂, Hexamethyldisilathiane (HMDST), PSCl₃/H₂O/Et₃N, polymer supported thionation agent and in situ thionation agent, such as e.g. P₂S₅/Na₂CO₃.

Preferred as the thionation agent for use in the process of the invention for the preparation of compounds of formula I is Lawesson's reagent.

The process of the present invention for the preparation of compounds of formula I is typically carried out in an organic solvent, which is preferably a nonpolar organic solvent selected from the group selected from the group consisting of toluene, n-hexane, benzene, pentane, chloroform, diethyl ether, 1,4-dioxane, carbon tetrachloride, and methylene chloride (dichloromethane).

The reaction is then performed at a temperature that is below or equal to the temperature of solvent reflux at atmospheric pressure, preferably at a temperature ranging from ambient temperature (20-30° C.) and the temperature of solvent reflux, preferably ranging from 50° C. and the solvent reflux at atmospheric pressure.

In a very preferred embodiment, the synthesis of 2-phenyl-4H-benzo[d][1,3]dioxine-4-thione involves first the reaction of benzaldehyde (2) with sulfuric acid to produce a diacetate (3), that is subsequently condensed with salicylic acid (1) under acidic conditions to produce the compound (4), which corresponds to the compound of formula II, wherein R₁, R₂, R₃ and R₄, as well as R₁′, R₂′, R₃′, R₄′ and R₅′ are H and X is O. The corresponding thionolactone (5) (2-phenyl-4H-benzo[d][1,3]dioxine-4-thione, i.e. the compound of formula 1, wherein R₁, R₂, R₃ and R₄, as well as R₁′, R₂′, R₃′, R₄′ and R₅′ are H and X is O) is obtained by reacting the compound (4) with Lawesson's reagent. An exemplary synthetic route for this very preferred embodiment is represented in the below Scheme 1 that reflects the procedure applied in Example 1 for the synthesis of 2-phenyl-4H-benzo[d][1,3]dioxine-4-thione.

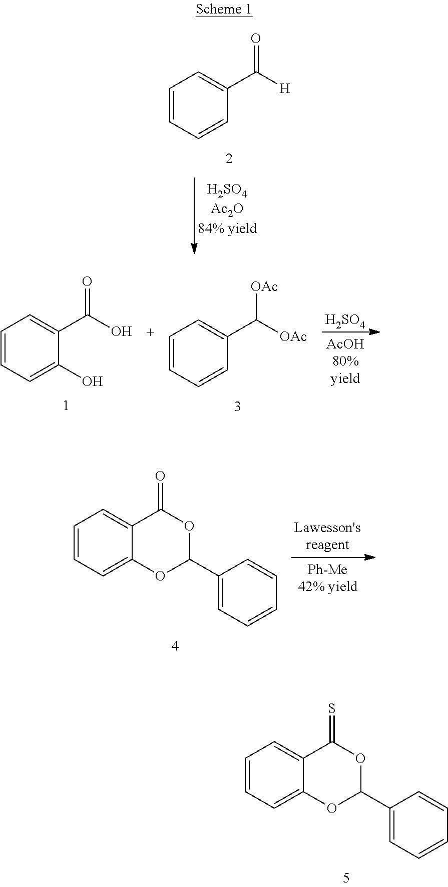

### Use of a Compound of Formula I as Comonomer

The present invention furthermore relates to the use of a thionolactone of formula I, as a comonomer for the preparation of copolymers. These copolymers include the copolymers of the present invention that will be described herein below.

### The Copolymer of the Present Invention

The present invention furthermore relates to a copolymer comprising the repeating unit of formula III

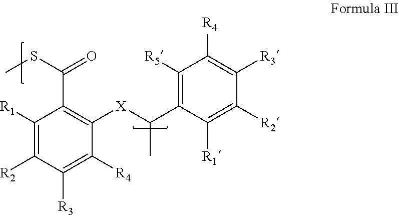

and at least another repeating unit.

In the compound of formula III, X, R₁, R₂, R₃ and R₄, as well as R₁′, R₂′, R₃′, R4′ and R₅′ are defined as described herein above for the compound of formula I.

In general, the at least another repeating unit of the copolymers of the present invention are derived from comonomers that are “more activated” in the sense of being useful as comonomers in a radical copolymerization reaction with the compounds of formula I.

In particular, the at least another repeating unit is derived from a comonomer selected from the group consisting of alkyl acrylates, N-alkylacrylamides, N, N-dialkylacrylamides, styrene and styrene derivatives, and acrylonitrile, or mixtures thereof.

“Alkyl” in the context of these comonomers means a linear or branched alkyl group comprising 1 to 12 carbons. Exemplary alkyl groups include, but are not limited to methyl, ethyl, n-propyl, iso-propyl, cyclo-propyl, n-butyl, iso-butyl, tert-butyl, n-pentyl, iso-pentyl, cyclo-pentyl, n-hexyl, cyclo-hexyl, n-heptyl, cyclo-heptyl, n-octyl, n-nonyl, n-decyl, n-undecanyl, n-dodecyl. Preferably, the linear or branched alkyl group comprises 1 to 6 carbons, more preferably 1 to 4 carbons, i.e. methyl, ethyl, n-propyl, iso-propyl, n-butyl, iso-butyl, tert-butyl, with methyl being particularly preferred.

Examples for alkyl acrylates useful as comonomers for the provision of copolymers of the present invention thus include, but are not limited to: methyl acrylate, ethyl acrylate, n-propyl acrylate, iso-propyl acrylate, n-butyl acrylate, iso-butyl acrylate, tert-butyl acrylate.

Examples for N-alkylacrylamides useful as comonomers for the provision of copolymers of the present invention thus include, but are not limited to: N-methylacrylamide, N-ethylacrylamide, N-n-propylacrylamide, N-iso-propylacrylamide, N-n-butylacrylamide, N-iso-butylacrylamide, N-tert-butylacrylamide.

Examples for N, N-dialkylacrylamides useful as comonomers for the provision of copolymers of the present invention thus include, but are not limited to: N,N-dimethylacrylamide, N,N-diethylacrylamide, N,N-di-n-propylacrylamide, N,N-di-iso-propylacrylamide, N, N-di-n-butylacrylamide, N, N-di-iso-butylacrylamide, N, N-di-tert-butylacrylamide.

“Styrene derivatives” as referred to herein means styrene that is substituted on the benzene moiety by one or more functional groups. Preferred styrene derivatives for the provision of copolymers of the present invention are styrene derivatives that are substituted in 4-position of the benzene moiety by a functional group (i.e. 4-substituted styrene derivatives). Functional groups for use in substituted styrene derivatives include, but are not limited to: p1 linear and branched alkyl comprising 1 to 4 carbons, i.e. methyl, ethyl, n-propyl, iso-propyl, n-butyl, iso-butyl, tert-butyl;
alkoxy comprising 1 to 4 carbons, i.e. methoxy, ethoxy, n-propyloxy, iso-propyloxy, n-butyloxy, iso-butyloxy, tert-butyloxy (e.g. 4-methoxystyrene);
phenoxy and phenoxy substituted with one or more functional groups;
halogen including F, Cl, Br, I;
perfluoralkyl including e.g. CF₃;
sulfonate (e.g. sodium-4-vinylbenzenesulfonate).

Preferably, the at least another repeating unit is selected from the group consisting of formula IV, V, VI below and mixtures thereof

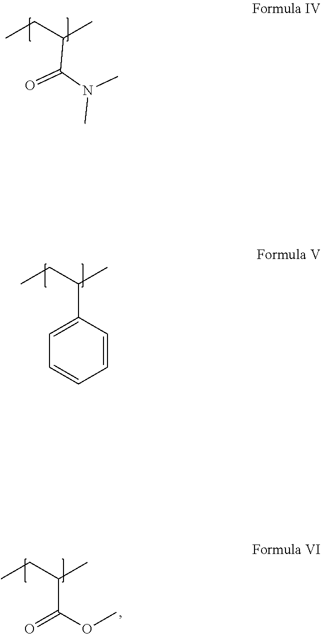

which are derived from N,N-dimethylacrylamide (Formula IV), styrene (Formula V) and methyl acrylate (Formula VI).

Comonomers such as vinyl acetate and ester derivatives thereof, carbazole, N-vinylpyrrolidone, ethylene, allylics, are “less activated” and therefore not suitable for use as comonomers for the provision of copolymers of the present invention. A similar assessment applies to methacrylates and methacrylamides, wherein the methyl group provides a stabilizing effect during radical polymerization that prevents copolymerization with the compounds of formula I.

The repeating unit of formula III, in particular the thioester linkage that forms part of the repeating unit III, introduces weak bonds into the copolymer that makes it suitable for degradation and functionalization.

### Process for the Preparation of a Copolymer of the Present Invention

The present invention furthermore relates to a process for the preparation of a copolymer of the present invention as described above. In the process of the present invention, a thionolactone of formula I as described herein is copolymerized with at least one other comonomer as described herein above in connection with the copolymers of the present invention. The copolymerization process of the present invention from a mechanistic point of view is a radical ring-opening copolymerization process, which introduces the thioester linkage as reflected in formula III within the polymer backbone.

In accordance with the description provided above for the copolymers of the present invention, the comonomer used in the copolymerization process of the present invention is selected from the group consisting of alkyl acrylates, N-alkylacrylamides, N,N-dialkylacrylamides, styrene and styrene derivatives, and acrylonitrile, or mixtures thereof.

Exemplary and preferred embodiments of the comonomer as described above in connection with the copolymers of the present invention also apply to the process for the preparation of the copolymers of the present invention. Hence, N,N-dimethylacrylamide, styrene, methyl acrylate and mixtures thereof are preferred comonomers for use in the process for the preparation of the copolymers of the present invention.

Comonomers such as vinyl acetate and ester derivatives thereof, carbazole, N-vinylpyrrolidone, ethylene, allylics, are “less activated” and therefore not suitable for use as comonomers in the process for the preparation of copolymer of the present invention. A similar assessment applies to methacrylates and methacrylamides, wherein the methyl group provides a stabilizing effect during radical polymerization that prevents copolymerization with the compounds of formula I.

The copolymerization process of the present invention is typically carried out in the presence of a radical initiator. While a range of well-known radical initiators can be used in the process for the preparation of the copolymers of the present invention, such as azo compounds including e.g. (AIBN) and azobisisobutyronitrile 1,1′-azobis(cyclohexanecarbonitrile) (ABCN); organic peroxide including e.g. dibenzoylperoxide (DBPO), di-tert-butylperoxide and methyl ethyl ketone peroxide; inorganic peroxide including e.g. peroxydisulfate salts such as e.g. Na₂S₂O₈, K₂S₂O₈ and (NH₄)₂S₂O₈. Typically and preferably however, the well-known azobisisobutyronitrile (AIBN) is used herein.

The copolymerization process of the present invention can be carried out as a bulk polymerization (i.e. in the absence of solvent).

Alternatively, the copolymerization process of the present invention can also be carried out in an organic solvent, which is typically a non-polar solvent, such as e.g. hydrocarbon solvents, such as e.g. pentane, hexane, benzene, toluene and ether solvents such as e.g. 1,4-dioxane, diethyl ether, tetrahydrofuran (THF), anisole; or a polar aprotic solvent such as e.g. dichloromethane (DCM), chloroform, ethyl acetate, acetone, dimethylformamide (DMF), dimethyl sulfoxide (DMSO); wherein anisole represents one preferred organic solvent for use in the copolymerization process of the present invention.

Alternatively, the copolymerization process of the present invention can also be carried out in a hydroalcoholic solvent mixture. Said hydroalcoholic solvent mixture comprises at least one alcohol and water, wherein the alcohol is selected from the group consisting of methanol, ethanol, n-propanol, isopropanol, n-butanol and mixtures thereof. Preferably, an ethanol/water mixture is used as the hydroalcoholic mixture in the copolymerization process of the present invention.

The ratio of the at least one alcohol and water in the hydroalcoholic solvent mixture (i.e. alcoholic component(s):water) ranges from 50:50 to 99:1 vol/vol, preferably 60:40 to 95:5 vol/vol, more preferably 70:30 to 90:10 vol/vol, particularly preferably 80:20 vol/vol. Thus, if for example an ethanol/water mixture is used as the hydroalcoholic solvent mixture, the ratio of ethanol and water in said mixture is particularly preferably 80:20 vol/vol.

Whether the copolymerization process of the present invention is to be carried out in an organic solvent mixture or in a hydroalcoholic solvent mixture can e.g. be decided on the basis of the solubility of the thionolactone in a respective solvent mixture.

The amount of comonomer used in the copolymerization process of the present invention typically ranges from 1 to 10 molar equivalents, based on the molar amount of the compound of formula I used. Preferably, the amount of comonomer ranges from 1.5 to 15 equivalents, more preferably from 2 to 12 molar equivalents, even more preferably from 3 to 9 molar equivalents, based on molar amount of the compound of formula I used. A higher relative molar amount of thionolactone in the mixture of thionolactone and comonomer increases the percentage of thioester linkages formed during copolymerization.

The amount of catalyst used in the copolymerization process of the present invention typically ranges from 0.1 mol-% to 15 mol-%, preferably from 0.2 mol-% to 10 mol %, even more preferably from 0.5 mol-% and 5 mol-%, such as e.g. 1 mol-%, 2 mol-%, 3 mol-% and 4 mol-%, based on the molar amount of the compound of formula I used in the copolymerization reaction.

The copolymerization process of the present invention is typically carried out at temperatures that range from ambient temperature to 120° C., preferably from 40° C. to 110° C., even more preferably from 60° C. to 100° C., even more preferably from 70° C. to 90° C., such as e.g. 80° C.

### Use of a Compound of Formula I as Comonomer to Impart Degradability Properties to a Copolymer Partially Made Therefrom

The present invention furthermore relates to the use of thionolactones of formula I as described herein as comonomer, to impart degradability properties to a copolymer partially made therefrom. The copolymer partially made therefrom is preferably a copolymer of the present invention as described herein above.

### Process for Degradation of a Copolymer

The present invention furthermore relates to a process for degradation of the copolymers of the present invention as described above. The degradation process of the present invention is carried out by reacting said copolymer with a degradation reagent selected from the group consisting of a base, an amine and an oxidant, preferably a base, in an organic or aqueous medium.

Depending on the degradation agent, the degradation process of the present invention can thus be carried out as hydrolysis (using a base as the degradation agent), aminolysis (using an amine as the degradation agent) or oxidative hydrolysis (using an oxidant as the degradation agent). The degradation of the copolymers of the present invention by means of the process described herein leads to the formation of smaller oligomers.

The degradation process of the present invention can be carried out in an organic medium comprising an organic solvent or a mixture of organic solvents. Organic solvents suitable for use in the degradation process of the present invention include common organic solvents such as e.g. alcohols, including e.g. methanol, ethanol, n-propanol, iso-propanol, n-butanol; ethers, including e.g. diethylether, methyl-tert-butylether, tetrahydrofuran (THF), chlorinated solvents, including e.g. dichloromethane (DCM) and chloroform; dimethylformamide (DMF); dimethylsulfoxide (DMSO); ethyl acetate; acetone and mixtures thereof.

Alternatively, the degradation process of the present invention can also be carried out in an aqueous medium, optionally further comprising an organic solvent or a mixture of organic solvents, wherein the organic solvent is preferably an alcohol selected from the group of methanol, ethanol, n-propanol and iso-propanol, n-butanol and mixtures thereof, with ethanol being particularly preferred. In such a hydroalcoholic medium (i.e. in a mixture of at least one alcohol and water), the amount of alcohol in the hydroalcoholic medium is typically at least 60%, preferably at least 70%, even more preferably at least 80% by weight.

While the addition of copolymer to the degradation reagent (comprised, preferably dissolved in an organic or aqueous medium) is possible for carrying out the degradation process of the present invention, the process is typically carried out by adding the degradation reagent to the organic or aqueous medium comprising the copolymer. The concentration of the copolymer in the medium is then typically within the range of 0.01 g/mL and 0.5 g/mL. The degradation reagent can be added neat or in organic or aqueous solution.

In the aminolytic degradation process of the present invention, amines such as ammonia, iso-propylamine or dimethylamine can be used as the degradation agent. The process is then preferably carried out in an organic medium such as e.g. methanol, tetrahydrofurane (THF) or dichloromethane (DCM). The skilled person is able to choose the amount of amine used in the degradation process in relation to the copolymer according to his/her needs taking into consideration his/her common general knowledge. The reaction mixture is typically stirred between 3 and 24 hours, preferably 6 to 18 hours, even more preferably 12 to 15 hours at ambient temperature.

In the oxidative hydrolytic degradation process of the present invention, oxidants such as oxone (KHSO₅·½KHSO₄·½K₂SO₄) can be used as the degradation agent. The process is then preferably carried out in an aqueous medium. The skilled person is able to choose the amount of oxidant used in the degradation process in relation to the copolymer according to his/her needs taking into consideration his/her common general knowledge. The reaction mixture is typically stirred between 3 and 24 hours, preferably 6 to 18 hours, even more preferably 12 to 15 hours at ambient temperature.

In the hydrolytic degradation process of the present invention, which is preferably used herein, common bases such as e.g. potassium hydroxide (KOH), sodium hydroxide (NaOH), lithium hydroxide (LiOH), ammonium hydroxide (NH₄OH), calcium hydroxide (Ca(OH)₂), magnesium hydroxide (Mg(OH)₂), sodium carbonate (Na₂CO₃), potassium carbonate (K₂CO₃), sodium hydrogen carbonate (NaHCO₃), potassium hydrogen carbonate (KHCO₃), sodium dihydrogen phosphate (NaH₂PO₄), potassium dihydrogen phosphate (KH₂PO₄) can be used, with potassium hydroxide and sodium hydroxide being preferred.

The base is typically added in aqueous, hydroalcoholic or alcoholic solution to a medium comprising the copolymer. The concentration of the base in solution is typically within the range of 1% wt/wt to 20% wt/wt, preferably 2% wt/wt to 10% wt/tw, such as e.g. 5% wt/wt. The skilled person is able to choose the amount of base used in the degradation process in relation to the copolymer according to his/her needs taking into consideration his/her common general knowledge. The resulting mixture is typically stirred between 3 and 24 hours, preferably 6 to 18 hours, even more preferably 12 to 15 hours at ambient temperature.

A preferred organic medium used in the hydrolytic degradation process using a base as the degradation reagent is THF or a mixture of THF and methanol, wherein in said mixture of THF and methanol, the ratio of THF: methanol is typically at least 60:40 vol/vol, preferably at least 70:30 vol/vol, more preferably at least 80:20 vol/vol, such as e.g. 90:10 vol/vol.

The hydrolytic degradation process of the present invention is then e.g. be carried out by dissolving the copolymer in tetrahydrofuran (THF), and a solution of the base, e.g. KOH in methanol, is added to the copolymer solution.

A preferred hydroalcoholic medium used in the hydrolytic degradation process using a base as the degradation reagent is a mixture of ethanol and water, wherein the ratio of ethanol: water is typically at least 60:40 wt/wt, preferably at least 70:30 wt/wt, more preferably at least 80:20 wt/wt, such as e.g. 80:20 wt/wt, 90:10 wt/wt and 95:5 wt/wt.

The hydrolytic degradation process of the present invention is then e.g. be carried out by dissolving the copolymer in an ethanol/water mixture (e.g. 80:20 wt/wt), and a solution of the base, e.g. KOH in methanol, is added to the aqueous copolymer solution.

### Use of a Compound of Formula I as Comonomer to Introduce Functionality into a Copolymer Partially Made Therefrom

The present invention furthermore relates to the use of a thionolactone of formula I, as comonomer to introduce functionality into a copolymer partially made therefrom. The copolymer partially made therefrom is preferably a copolymer of the present invention as described herein above.

The functionality introduced by the compound of formula I used as comonomer into a copolymer partial made therefrom in form of a thioester linkage makes the copolymer suitable for functionalization, e.g. when treated with a degradation agent selected from the group consisting of a base, an amine and an oxidant, as described above. Smaller oligomers obtained from such treatment are functionalized with —SH and —COOH on each end of the oligomer due to thioester cleavage.

### Process for Functionalization of a Copolymer

The present invention furthermore relates to a process for functionalization of the copolymers of the present invention as described above. The functionalization process of the present invention is carried out by reacting said copolymer with a degradation reagent selected from the group consisting of a base, an amine and an oxidant, preferably a base, in an organic or aqueous medium.

The process details for the functionalization process of the present invention are the same as described above for the degradation process of the present invention.

The functionalization process of the present invention leads to the provision of smaller oligomers obtained from such treatment are functionalized with —SH and —COOH groups on each end of the oligomer due to thioester cleavage.

### Oligomers

The present invention furthermore relates to oligomers obtainable by the process for degradation of a copolymer as described above or by the process for functionalization of a copolymer as described above.

The following examples are intended to illustrate the present invention without meaning to limit its scope.

### EXPERIMENTAL PART

### Example 1: Synthesis of the Thionolactone 2-phenyl-4H-benzo[d][1,3]dioxine-4-thione (Formula I).

### 1.1 Synthesis of Phenylmethylene Diacetate

To a solution of benzaldehyde (10.6 g) in acetic anhydride (100 mL) was added dropwise a solution of H₂SO₄ (160 mg) in acetic anhydride (20 mL). Stirring was then continued for 1 hour and then the mixture poured into an ice-H2O mixture. The mixture was extracted with EtOAc (3×100 mL) and then the combined organic phases were washed with H₂O (×2) and sat. NaHCO₃. The solvent was removed by rotary evaporation (60° C.). Residual acetic anhydride was then removed under high vacuum at 55° C. to give phenylmethylene diacetate (17.4 g, 84%) as a colorless solid.

¹H NMR (CDCl₃, 400 MHZ): δ 2.12 (s, 6H), 7.38-7.43 (m, 3H), 7.49-7.54 (m, 2H), 7.68 (s, 1H).

¹³C NMR: (CDCl₃, 100 MHZ): δ 20.5, 89.4, 126.4, 128.3, 129.4, 135.3, 168.4.

1.2 Synthesis of 2-phenyl-4H-benzo[d][1,3]dioxin-4-one (Formula II)

A mixture of salicylic acid (4.8 g), phenylmethylene diacetate (7.25 g) obtained as described above, acetic acid (3 mL) and H₂SO₄ (0.01 mL) were placed in a distillation apparatus and then held at 27 mbar and heated to 70° C. Once the distillation of acetic acid halted, the temperature was then slowly raised to 110° C. over a period of 2 hours. The reaction was then cooled and the residual solid subsequently taken up in Et₂O and washed with sat. NaHCO₃ and sat. NaHSO₃. The solvent was then removed by rotary evaporation to give 2-phenyl-4H-benzo[d][1,3]dioxin-4-one (6.3 g, 80%) as a tan solid.

¹H NMR (CDCl₃, 400 MHZ): δ 6.53 (s, 1H), 7.07-7.14 (m, 1H), 7.17-7.25 (m, 1H), 7.43-7.51 (m, 3H), 7.55-7.69 (m, 3H), 8.01-8.06 (m, 1H).

¹³C NMR: (CDCl₃, 100 MHZ): δ 100.5, 114.6, 116.9, 123.6, 126.6, 128.7, 130.3, 130.4, 134.1, 136.5, 158.2, 161.9.

1.3 Synthesis of 2-phenyl-4H-benzo[d][1,3]dioxine-4-thione (Formula I)

A solution of 2-phenyl-4H-benzo[d][1,3]dioxin-4-one (8 g) obtained as described above and Lawesson's reagent (5.7 g, 0.4 equiv.) in toluene (400 mL) was refluxed for 24 hours. The reaction was then cooled and pulled through plug of silica using excess toluene. The solvent was then removed by rotary evaporation and the residue purified by flash column chromatography (20% EtOAc in pentane). The residue was then recrystallized from a DCM/pentane to give 2-phenyl-4H-benzo[d][1,3]dioxine-4-thione (3.6 g, 42% yield) as an orange solid.

¹H NMR (CDCl₃, 400 MHZ): δ 6.44 (s, 1H), 7.05-7.20 (m, 2H), 7.46-7.52 (m, 3H), 7.55-7.63 (m, 1H), 7.66-7.73 (m, 2H), 8.29-8.36 (m, 1H).

¹³C NMR: (CDCl3, 100 MHZ): δ 101.0, 117.0, 123.1, 123.8, 126.9, 128.7, 130.6, 133.0, 133.6, 136.2, 153.3, 202.7.

### Example 2: Copolymerization of 2-phenyl-4H-benzo[d][1,3]dioxine-4-thione (Formula I) with Different Comonomers

### 2.1 Copolymerization in Bulk

### 2.1.1 Dimethylacrylamide

### 21A—10% mol of Thionolactone (Relative to the Mixture of Thionolactone and N,N-dimethylacrylamide)

2-Phenyl-4H-benzo[d][1,3]dioxine-4-thione (100 mg) obtained as described above, N,N-dimethylacrylamide (368 mg, 9 equiv.) and AIBN (6.8 mg, 1 mol %) were charged into a headspace vial equipped with a magnetic stir bar. The vial was then sealed and degassed for 30 minutes using argon. The vial was then submerged in a pre-heated oil bath held at 80° C. After 24 hours, the solid was taken up in CHCl₃ (3 mL) and then precipitated into pentane (50 mL), followed by centrifugation. The isolated solid was then allowed to dry under high vacuum at room temperature.

¹H NMR (CDCl₃, 400 MHZ): 16.2% thioester linkages

### 2.1.2 Styrene

### 21B—10% mol of Thionolactone (Relative to the Mixture of Thionolactone and Styrene)

2-Phenyl-4H-benzo[d][1,3]dioxine-4-thione (100 mg) obtained as described above, styrene (387 mg, 9 equiv.), AIBN (6.8 mg, 1 mol %) and anisole (3 mL) were charged into a headspace vial equipped with a magnetic stir bar. The vial was then sealed and degassed for 30 minutes using argon. The vial was then submerged in a pre-heated oil bath held at 80° C. After 72 hours, the mixture was precipitated into Et₂O (50 mL) followed by centrifugation. The isolated solid was then allowed to dry under high vacuum at room temperature.

¹H NMR (CDCl₃, 400 MHZ): 10.4% thioester linkages

GPC (THF, Calibration with PS standards): Mn=12700, PDI=2.4

### 2.1.3 Methyl Acrylate

### 21C—10% mol of Thionolactone (Relative to the Mixture of Thionolactone and Methyl Acrylate)

2-Phenyl-4H-benzo[d][1,3]dioxine-4-thione (100 mg) obtained as described above, methyl acrylate (320 mg, 9 equiv.), AIBN (6.8 mg, 1 mol %) and anisole (3 mL) were charged into a headspace vial equipped with a magnetic stir bar. The vial was then sealed and degassed for 30 minutes using argon. The vial was then submerged in a pre-heated oil bath held at 80° C. After 72 hours, the mixture was precipitated into pentane (50 mL) followed by centrifugation. The isolated solid was then allowed to dry under high vacuum at room temperature.

¹H NMR (CDCl₃, 400 MHZ): 16.9% thioester linkages

In bulk copolymerization, the thioester linkages are higher than the theoretical values (which means calculated values based on the initial ratio between the thionolactone and the comonomer), except when the bulk copolymerization is carried out with styrene.

2.2 Copolymerization with DMAA in Organic Solvent

### 22A—8.7% mol of Thionolactone (Relative to the Mixture of Thionolactone and N,N-dimethylacrylamide)

2-Phenyl-4H-benzo[d][1,3]dioxine-4-thione (200 mg) obtained as described above, N,N-dimethylacrylamide (858 mg, 10.48 equiv.), AIBN (47 mg, 3 mol %) and anisole (5 mL) were charged into a headspace vial equipped with a magnetic stir bar. The vial was then sealed and degassed for 30 minutes using argon. The vial was then submerged in a pre-heated oil bath held at 80° C. After 20 hours, the mixture was precipitated into Et₂O (100 mL) followed by centrifugation. The isolated solid was then allowed to dry under high vacuum at room temperature.

¹H NMR (CDCl₃, 400 MHZ): 7.5% thioester linkages

GPC (THF, Calibration with PS standards): Mn=5300, PDI=1.8

### 22B—10% mol of Thionolactone (Relative to the Mixture of Thionolactone and N,N-dimethylacrylamide)

2-Phenyl-4H-benzo[d][1,3]dioxine-4-thione (100 mg) obtained as described above, N,N-dimethylacrylamide (368 mg, 9 equiv.), AIBN (6.8 mg, 1 mol %) and anisole (2.5 mL) were charged into a headspace vial equipped with a magnetic stir bar. The vial was then sealed and degassed for 30 minutes using argon. The vial was then submerged in a pre-heated oil bath held at 80° C. After 3 hours, the mixture was precipitated into pentane (50 mL) followed by centrifugation. The isolated solid was then allowed to dry under high vacuum at room temperature.

¹H NMR (CDCl₃, 400 MHZ): 8.7% thioester linkages

GPC (THF, Calibration with PS standards): Mn=7200, PDI=2.1

### 22C—25% mol of Thionolactone (Relative to the Mixture of Thionolactone and N,N-dimethylacrylamide)

2-Phenyl-4H-benzo[d][1,3]dioxine-4-thione (300 mg) obtained as described above, N,N-dimethylacrylamide (368 mg, 3 equiv.), AIBN (24 mg, 3 mol %) and anisole (4 mL) were charged into a headspace vial equipped with a magnetic stir bar. The vial was then sealed and degassed for 30 minutes using argon. The vial was then submerged in a pre-heated oil bath held at 80° C. After 24 hours, the mixture was precipitated into Et₂O (100 mL) followed by centrifugation. The isolated solid was then allowed to dry under high vacuum at room temperature.

¹H NMR (CDCl₃, 400 MHZ): 22% thioester linkages

GPC (THF, Calibration with PS standards): Mn=6400, PDI=1.7

Conclusion on the influence of the molar ratio of thionolactone and copolymer in the solvent polymerization: One can observe a good correlation between the thioester linkages in the copolymers and the initial amount of thionolactone in the mixture with the comonomer, indicating that the comonomers react at the same speed.

2.3 Copolymerization with DMAA in Water and Water/Ethanol Mixture

### 23A—10% mol of Thionolactone (Relative to the Mixture of Thionolactone and N,N-dimethylacrylamide)

2-Phenyl-4H-benzo[d][1,3]dioxine-4-thione (150 mg) obtained as described above, N,N-dimethylacrylamide (552 mg, 9 equiv.), AIBN (5.1 mg, 0.5 mol %) and an ethanol/H₂O 80/20 vol/vol mix (1053 mg) (to give approx. 40 wt % solids) were charged into a headspace vial equipped with a magnetic stir bar. The vial was then sealed and degassed for 30 minutes using argon. The vial was then submerged in a pre-heated oil bath held at 80° C. After 20 hours, the mixture was precipitated into pentane (50 mL) followed by centrifugation. The isolated solid was then allowed to dry under high vacuum at room temperature.

¹H NMR (CDCl₃, 400 MHZ): 12% thioester linkages

GPC (THF, PS standards): Mn=5500, PDI=1.9

### Example 3: Degradation of Copolymers in Basic Conditions

### 3.1 Degradation of Copolymer Produced in Organic Solvent

A sample of the copolymer 22A (200 mg) was dissolved in THF (9 mL) and 5% wt/wt KOH in MeOH (1 mL) and stirred overnight. The solvents were then removed by rotary evaporation.

GPC (THF, PS standards): Mn=900, PDI=2.5

A sample of the copolymer 22B (150 mg) was dissolved in THF (9 mL) and 5% wt/wt KOH in MeOH (1 mL) and stirred overnight. The solvents were then removed by rotary evaporation.

GPC (THF, PS standards): Mn=700, PDI=2.6

A sample of the copolymer 22C (150 mg) was dissolved in THF (9 mL) and 5% wt/wt KOH in MeOH (1 mL) and stirred overnight. The solvents were then removed by rotary evaporation.

GPC (THF, PS standards): Mn=300, PDI=3.2

The molecular weights of PDMA (poly-N,N-dimethylacrylamide) obtained by degradation of the copolymers produced in organic solvent evolve linearly with the amount of thioester linkages and are in fairly good agreement with the expected theoretical masses.

### 3.2 Degradation of Copolymer Produced in Ethanol/Water Mixture

A sample of the copolymer 23A (100 mg) was dissolved in an ethanol/water mixture (80/20 wt/wt) (9 mL) and 5% wt/wt KOH in MeOH (1 mL) and stirred overnight. The solvents were then removed by rotary evaporation.

5 GPC (THF, PS standards): Mn=400, PDI=2.6

1. A thionolactone of formula I 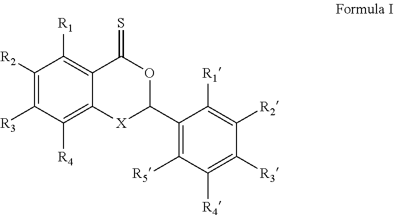
wherein:
X is either a heteroatom selected from the group consisting of O and S, or NAlk, with Alk being a linear or branched alkyl group comprising 1 to 6 carbons,
R₁, R₂, R₃ and R₄, as well as R₁′, R₂′, R₃′, R₄′ and R₅′ are independently selected from the group consisting of H, halogen, hydroxyl (—OH), thio (—SH), nitro group (—NO₂), amines (—NH₂), ammonium (—NH₄⁺), sulfate (—SO₄⁻), sulfonate (—SO₃⁻), phosphate (—PO₄²⁻), phosphonate (—PO₃²⁻), and hydrocarbyl comprising from 1 to 50 carbon atoms optionally substituted by one or more heteroatom containing groups; and/or interrupted by one or more heteroatoms or heteroatom containing groups; and/or optionally in the form of a cycle, aromatic or not, that is optionally interrupted by one or more heteroatoms or heteroatom containing groups; wherein heteroatom means O, N or S.

2. The thionolactone of formula I according to claim 1, wherein X is O.

3. The thionolactone of formula I according to claim 1, wherein R₁, R₂, R₃ and R₄, as well as R₁′, R₂′, R₃′, R₄′ and R₅′ are independently selected in the group consisting of —H, —F, —Cl, —Br, hydroxyl (—OH), thio (—SH), nitro group (—NO₂), amines (—NH₂), ammonium (—NH₄⁺), sulfate (—SO₄⁻), sulfonate (—SO₃⁻), and hydrocarbyl comprising from 1 to 50 carbon atoms substituted by one or more heteroatom containing groups selected from —OY, —NHY, —NY₂, —SY; and/or interrupted by one or more heteroatoms or heteroatom containing groups selected from —O—, —NY—, —S—, wherein Y means H or branched or linear alkyl comprising 1 to 12 carbons.

4. The thionolactone of formula I according to claim 1, wherein one of R₁, R₂, R₃, R₄, R₁′, R₂′, R₃′, R₄′ or R₅′ is a polyethylene glycol group of formula—(CH₂—CH₂—O)ₙ—H with n being an integer varying from 1 to 25.

5. The thionolactone of formula I according to claim 1, wherein at least R₁, R₁′, and R₅′ are H.

6. The thionolactone of formula I according to claim 1, wherein R₁, R₂, R₃ and R₄, as well as R₁′, R₂′, R₃′, R₄′ and R₅′ are H.

7. A process for the preparation of a thionolactone of formula I as defined in claim 1, the process comprising:
reacting a compound of formula II with a thionation agent 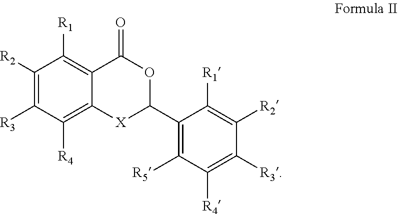
wherein X, R₁, R₂, R₃ and R₄, as well as R₁′, R₂′, R₃′, R₄′ and R₅′ are as defined in claim 1.

8. The process according to claim 7, wherein the thionation agent is selected from the group consisting of Lawesson's reagent (2,4-bis(4-methoxyphenyl)-1,3,2,4-dithiadiphosphetane 2,4-disulfide), Davy's reagent (2,4-Bis(methylthio)-2,4-dithioxo-1,3,2,4-dithiadiphosphetane, (CH₃S)₂P₂S₄)), Curphey's reagent (hexamethyldisiloxane (HMDO)/phosphorus pentasulfide (P₄S₁₀)), Kaushik's reagent (P₄S₁₀/Al₂O₃), Bernthsen reagent (S₈/I₂), Heimgartner's reagent (2,4-bis(4-methylphenylthio)-1,3,2λ5,4λ5-dithiadiphosphetane-2,4-dithione), Jan Bergman Reagent, Belleau reagent (2,4-bis(4-phenoxyphenyl)-1,3,2,4-dithiadiphosphetane 2,4-disulfide), Japanese reagent (2,4-bis(phenylthio)-1,3,2,4-dithiadiphosphetane 2,4-disulfide), H₂S, CS₂, R₂PSX, (Et₂Al)₂S, NaSH, TMS₂S, thiourea/Ru(III)/Al₂O₃, benzyltriethylammonium tetrathiomolybdate, thioacyl-N-pthalimides, elemental sulfur, aqueous ammonium sulfide, SiS₂, Hexamethyldisilathiane (HMDST), PSCl₃/H₂O/Et₃N, polymer supported thionation agent and in situ thionation agent, P₂S₅/Na₂CO₃.

9. A method for preparing copolymers, the method comprising providing a thionolactone of formula I as defined in claim 1 as a, as comonomer.

10. A copolymer comprising the repeating unit of formula III 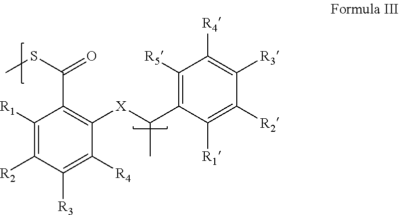
wherein X, R₁, R₂, R₃ and R₄, as well as R₁′, R₂′, R₃′, R₄′ and R₅′ are as defined in claim 1, and
at least another repeating unit.

11. The copolymer according to claim 10, wherein the at least another repeating unit is derived from a comonomer selected from the group consisting of alkyl acrylates, N-alkylacrylamides, N,N-dialkylacrylamides, styrene and styrene derivatives.

12. The copolymer according to claim 10, wherein the at least another repeating unit is selected from the group consisting of formula IV, V, VI, and mixtures thereof, wherein formula IV, V, VI are:

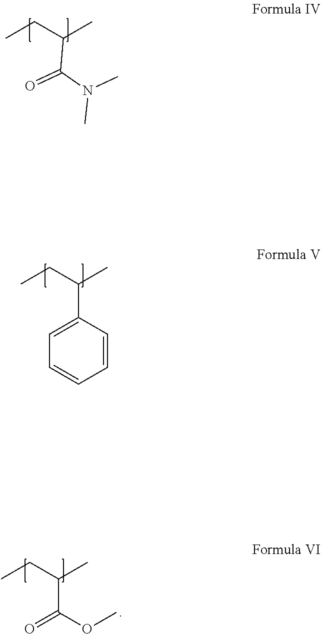

13. A process for preparing a copolymer, the process comprising copolymerizing a thionolactone of formula I with at least one other comonomer as defined in claim 11.

14. The thionolactone of formula I as defined in claim 1, wherein the thionolactone of formula I is configured to impart degradability properties to a copolymer partially made therefrom.

15. A process for degrading the copolymer as defined claim 10, the process comprising reacting the copolymer with a degradation reagent selected from the group consisting of a base, an amine and an oxidant in an organic or aqueous medium.

16. The thionolactone of formula I as defined in claim 1, wherein the thionolactone of formula I is configured to introduce functionality into a copolymer partially made therefrom.

17. A process for functionalizing the copolymer as defined in claim 10, the process comprising reacting the copolymer with a degradation reagent selected from the group consisting of a base, an amine and an oxidant in an organic or aqueous medium.

18. Oligomers obtainable by the process for degrading a copolymer as defined in claim 15.

19. Oligomers obtainable by the process for functionalizing a copolymer as defined in claim 17.

20. A process for preparing a copolymer, the process comprising copolymerizing a thionolactone of formula I with at least one other comonomer as defined in claim 12.
